(19) United States
(12) Patent Application Publication
(10) Pub. No.: US 2002/0019707 A1
(43) Pub. Date: Feb. 14, 2002

(54) Glucose metering system

(76) Inventors: Alan M. Cohen, Newton, MA (US); Marcelo R. Risk, Melrose, MA (US); Wayne Y. Menzie, Medford, MA (US); Keith N. Knapp II, Townsend, MA (US); John Schafer, Hampton, NH (US)

Correspondence Address: DALY, CROWLEY & MOFFORD, LLP, SUITE 101, 275 TURNPIKE STREET, CANTON, MA, 02021-2310, US

(21) Appl. No.: 09/891,572

(22) Filed: Jun. 25, 2001

Related U.S. Application Data
(60) Provisional application No. 60/213,913, filed on Jun. 26, 2000.

(51) Int. Cl. G01N 33/00

(52) U.S. Cl. 702/30; 436/95; 422/68.1; 422/67

(57) ABSTRACT

A glucose meter includes means for processing a blood sample to provide a glucose measurement and a communication interface to send and receive data including the glucose measurement to a remote processing center. The processing center collects and processes a plurality of glucose measurements to provide a modified method for processing a blood sample in order to provide an adjusted glucose measurement. In one embodiment, the modified blood sample processing method is a function of a permissible glucose deviation for the patient and in another embodiment, the modified blood sample processing method is a function an aging factor indicative of test strip variations over time.

### CROSS-REFERENCE TO RELATED APPLICATIONS

This application claims priority under 35 U.S.C. § 119e to U. S. Provisional Patent Application No. 60/213,913 filed Jun. 26, 2000 which application is hereby incorporated by reference herein in its entirety.

### STATEMENTS REGARDING FEDERALLY SPONSORED RESEARCH

Not applicable.

### FIELD OF THE INVENTION

This invention relates generally to convenient strip-based glucose test meters for aiding diabetic patients and more particularly to systems and techniques for linking a personal glucose meter, such as is used by a person with diabetes, to a central processing center, for the purpose of increasing the quality of glucose measurements.

### BACKGROUND OF THE INVENTION

In the control of diabetes, it is often desirable to perform glucose tests to record glucose readings to help manage diabetic treatment. Typically, a diabetic patient is instructed to periodically perform a personal glucose test and to maintain a log of the glucose readings and other relevant information. Such tests performed by a patient may be referred to as “personal glucose tests” performed on a “personal glucose meter” to differentiate them from higher-quality but less convenient glucose tests that must be performed in specialized laboratories. More specifically, the patient is instructed to measure their blood glucose at scheduled times and record the glucose reading, and depending on the readings, the amount of insulin or other medications required by the patient to maintain normal blood glucose levels may be determined. Furthermore, physicians must review the hand recorded data to adjust the patient's diabetic therapy. It has been observed that most patients fail to perform the personal glucose test at the scheduled time either performing the tests late or failing to take the test altogether, thereby adding inaccuracy to the proper treatment of diabetes.

Personal glucose tests require that a drop of blood be dripped onto a strip, then inserted into the glucose meter. The strip contains chemicals that react with the blood, and the meter reads this reaction by various means. Each lot of strips that is manufactured is slightly different, which is a source of inaccuracy in testing and which further adds to the improper treatment of diabetes.

It has also been observed that glucose readings on personal machines usually vary within twenty percent of the readings that,,would be obtained from a laboratory test. Furthermore, due to various factors, for example, too little blood on the strip, an occasional reading can be excessively off further adding to the improper treatment of diabetes.

In addition to performing the personal glucose test at the scheduled time, it is also important to know when the reading was taken with relationship to any meals eaten by the patient being tested. With today's busy schedule, most patients eat at irregular intervals which further adds to the complexity of proper treatment of diabetes.

### SUMMARY OF THE INVENTION

It is an object of the invention to collect various data regarding a patient's use of a personal glucose meter to improve the proper treatment of diabetes.

It is a further object of the invention to improve the accuracy of glucose measurements.

According to the invention, a glucose meter includes means for processing a blood sample on a test strip to provide a glucose measurement and a communication interface to send data including glucose measurements to a processing center and to receive data from the processing center. The processing means is responsive to data received from the processing center for varying the processing of a blood sample in order to provide an adjusted glucose measurement. With this arrangement, blood sample processing methods are modified in response to analysis of prior glucose readings by the processing center. The result is a highly accurate glucose reading which has been compensated for inaccuracies. Illustrative data received from the processing center for modifying blood sample processing are an aging factor indicative of test strip variations over time and a likely glucose range for the patient.

Also described is a glucose metering system including the glucose meter and a processing system coupled to the glucose meter and including means for collecting a plurality of glucose measurements from the meter and for processing the glucose measurements to provide a modified blood sample processing method. The modified blood sample processing method may be implemented by the glucose meter or by the processing center and the adjusted glucose measurement sent to the glucose meter for display.

According to a further aspect of the invention, a glucose metering system includes a glucose meter for processing a blood sample on a test strip having a response curve and a remote processing system in communication with the glucose meter. The processing system includes means for collecting data including a plurality of glucose measurements from the glucose meter and a description of the test strip response curve. The processing system processes the data to provide an aging factor indicative of variations in the test strip over time. The aging factor is used to provide an adjusted glucose measurement which is compensated for the effects of test strip variations over time, thereby providing an accurate glucose measurement. In one embodiment, the description of the test strip response curve is a single number code. And, in an alternative embodiment, the test strip response curve description is a polynomial equation.

Also described is a method for providing a glucose measurement including the steps of sending a plurality of glucose measurements of a patient from a glucose meter to a remotely located processing center and evaluating the glucose measurements to derive a likely glucose range for the patient. Further glucose measurements of the patient are processed in response to the likely glucose range in order to determine whether the measurement is an accurate measurement or an artifact. With this arrangement, inaccurate glucose measurements are discarded and prevented from erroneously impacting patient care.

### BRIEF DESCRIPTION OF THE DRAWINGS

The foregoing features of this invention, as well as the invention itself, may be more fully understood from the following description of the drawings in which:

FIG. 1 is a diagram of a glucose meter according to the invention;

FIG. 2 is a diagram of a glucose metering system including a plurality of glucose meters of the type shown in FIG. 1, a telemetry link, and a processing center according to the invention;

FIG. 3 is a flow diagram illustrating a process performed by the processing center for deriving a patient's likely glucose range;

FIG. 4 is a flow diagram illustrating a process performed by the processing center for deriving an aging factor indicative of test strip variations over time;

FIG. 5 is a more detailed diagram of the glucose metering system of FIG. 2; and

FIG. 6 shows an illustrative response curve of a glucose test strip.

### DETAILED DESCRIPTION OF THE INVENTION

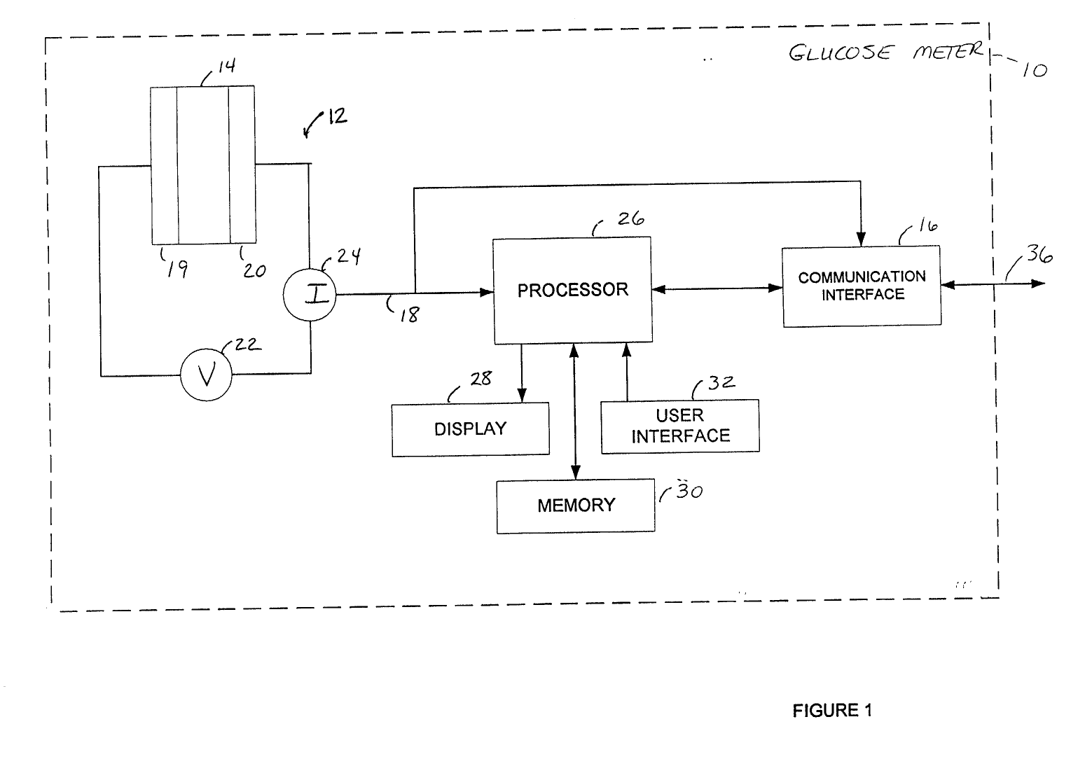

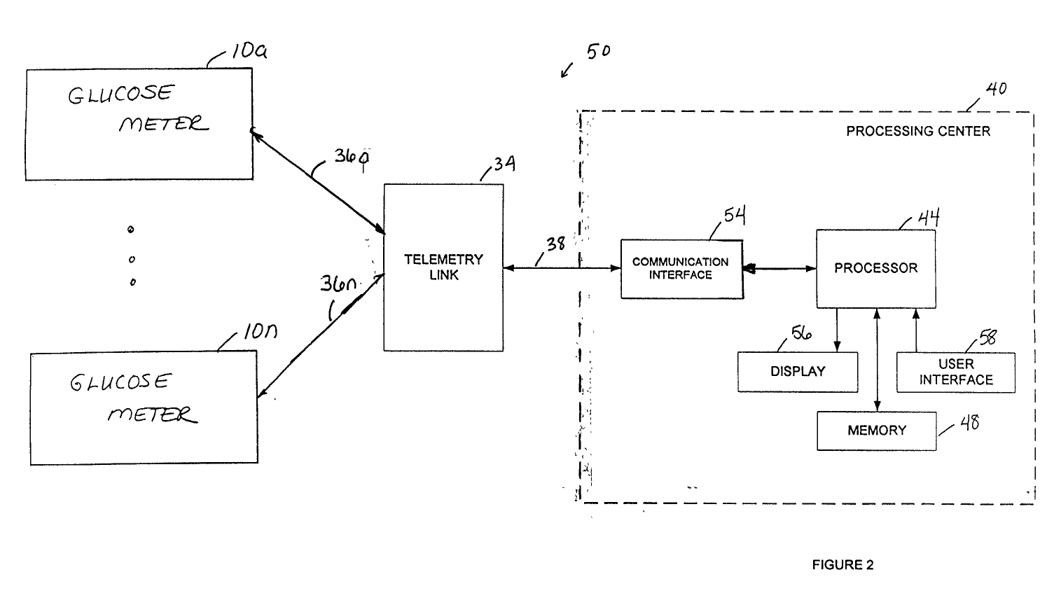

Referring to FIG. 1, a glucose meter 10 according to the present invention is adapted to operate in conjunction with a processing system, or center 40 (FIG. 2) in order to improve the accuracy of glucose measurements and the proper treatment of diabetes. Data collected at the glucose meter 10, such as glucose measurements and other glucose measurement related data, is transmitted for processing by the processing center. Data processing by the processing center yields modified methods with which a blood sample is processed to provide a highly accurate glucose measurement and/or instructions to the user, or patient regarding care, as will be described.

The glucose meter 10 includes a test circuit 12 to test a sample of blood dripped, or otherwise placed on a test strip 14. The strip 14 contains chemicals that react with the blood, and the meter reads this reaction by various means. The blood may be mixed with other materials to increase the efficacy of the measurement.

There are two basic ways in which the glucose level can be discerned from a blood sample, using electrical properties or using photometric/spectral properties. When using electrical properties, a voltage from a source 22 is maintained across the blood sample via electrodes 19 and 20, and the resulting current is measured by a current sensor 24 to provide a signal 18 indicative of the blood glucose level. In the case where photometric/spectral properties of the blood sample are measured, light from a light source 19 is passed through, or reflected from, the blood sample, and the resulting light spectra is measured, usually using a photodetector 20 that emits a voltage or current signal 18 in response to light.

The above-mentioned techniques have in common that the blood glucose level is measured with a test circuit 12 that provides an electrical signal 18, in the form of a current or voltage, which is indicative of the level of glucose in the blood sample. The voltage or current signal 18 which is generated using properties of the blood sample provides a “raw glucose measurement”. This raw measurement may optionally be processed by a digital processing system, or simply processor 26 to produce a “preliminary glucose measurement” based on general information about the properties of the meter and strips, generally expressed in units of milligrams/deciliter or millimoles/liter. Optionally, the raw or preliminary glucose measurement may be processed using a processing method, or algorithm implemented by the processor 26 in order to provide an “adjusted glucose measurement” based on information developed by the processing center. In this case, the processor 26 receives the signal 18, generally in digital form, and processes the digital signal to adjust the measurement to correct for inaccuracies, such as those caused by variations in the manufacture of the test strip.

A user interface 32 permits a patient to input the lot grade or other characterizing information about the test strip into the meter 10. Additional information may be supplied through the interface 32, such as the timing of a meal relative to the timing of a glucose test and/or the meal content for use by the processing center, as described below.

A display 28 is provided for displaying to the patient the glucose measurement in the form of a preliminary glucose measurement and/or an adjusted glucose measurement. The display may additionally operate in conjunction with the user interface 32 to display information input by the patient and optionally to display menu or prompt information to further assist in the accurate inputting of information. The display 28 may also be used to convey to the patient instructions or other messages from a physician or the processing center, as will be described.

Memory 30 is coupled to the processor 26 for storing glucose related data pertaining to glucose measurements, such as raw, preliminary, and/or adjusted glucose measurements, lot grade, and food intake information. The glucose meter 10 contains a clock circuit so that the data relating to a particular glucose measurement can be stored in association with a time and date stamp.

The glucose meter 10 further includes a communication interface 16 to send glucose measurements and other data relating to glucose measurements to the processing center 40. More generally, the communication interface 16 can send data to the processing center and receive data from the processing center and may take various forms, such as a modem.

Referring also to FIG. 2, a glucose metering system 50 according to the present invention includes a processing center 40 having means for collecting and processing data relating to glucose measurements from one or more glucose meters 10a-10n, each of the type shown in FIG. 1 The processing center 40 is coupled to the glucose meters 10a-10n through a telemetry link 34 and connections 36a-36n and 38, as shown. The telemetry link and connections might include a telephone line, an Internet connection, a two-way pager system (e.g., offered by SkyTel), or any similar communication link. For example, the link and connections may be implemented with various types of hard-wire or wireless media and may further include one or more public or private networks, such as a local area network (LAN) or a wide area network (WAN) which may be part of the Internet. The telemetry link and connections may also include a radio frequency (RF) connection which eliminates the need for a hard-wire connection to a telephone line.

Data relating to glucose measurements may be collected at the central processing center 40 using an interface based on a World-Wide Web site and a web browser. The web software can guide the user through the various steps of attaching the meter to the computer by means of a serial (i.e., RS-232) connection or similar connection, downloading data from the meter to the web site for storage in a database, and entry and download of other useful data, such as strip lot grade or number, patient name, patient ID number, and so forth.

Preferably, the glucose meter 10 is a relatively small, battery-powered, portable unit which is kept with the patient to permit glucose readings to be taken conveniently. The processing center 40 typically is located some distance from the patient and may be at a physician's office or other facility. Although the actual distance between the processing center and the location of the glucose meters varies and may in fact be a relatively short distance, the processing center can be described as being located remotely with respect to the glucose meter since during typical use, the glucose meter is not located at the processing center.

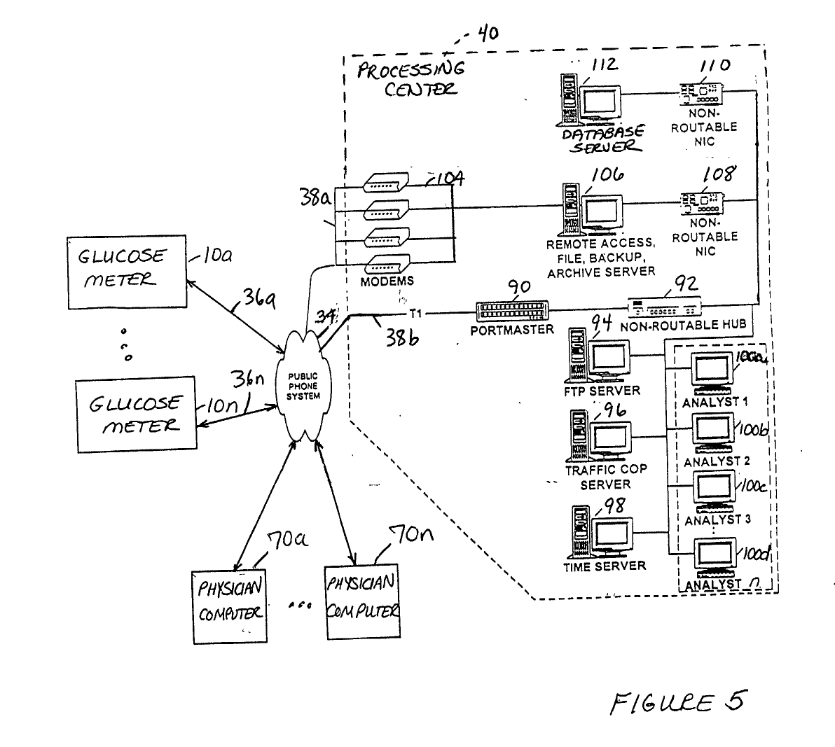

The processing center 40 includes a processor 44, a memory 48 in the form of a database 48, a communication interface 54 coupled to telemetry link 34 for communicating data between one or more glucose meters and the processing center, a display 56, and a user interface 58. One illustrative implementation of the processing center 40 is shown in FIG. 5 and described below. However, it will be appreciated that the functional elements of the processing center may be implemented by various configurations of both hardware and software elements.

The processor 44 implements processes, or algorithms by which data collected from one or more glucose meters 10a-10n is analyzed and evaluated in order to provide a modified method for processing a blood sample and/or patient instructions, as will be described. Illustrative data sent to the processing center includes one or more glucose measurements, which may be raw measurements and/or preliminary measurements, food consumption information, test strip characterizing information, test times, and patient identification. In the case where the glucose meter 10 does not process the raw measurement to provide an adjusted glucose reading, the raw measurement and/or a preliminary measurement is processed at the processing center for conversion to an adjusted glucose reading. The adjusted glucose reading may be returned to the meter 10 for display to the user on the glucose meter display 28. As an alternative, the glucose reading may remain at the processing center, for example for viewing over a web browser.

One advantage to processing the raw or preliminary glucose measurement at the processing center 40 to provide an adjusted glucose measurement is that more complex processing algorithms may be used than can be performed in the portable meter 10 given the typical processing power of the processor 26. Further, if algorithms for converting the raw or preliminary measurement into an adjusted measurement are modified from time to time, it may be easier to implement the modification (i.e., to update the software) at the processing center 40 than to update many portable meters 10a-10n. However, algorithms implemented in the glucose meters 10a-10n may be updated by downloading a new algorithm from the processing center via the telemetry link 34.

Further, it should be appreciated that connecting to the processing center 40, sending raw or preliminary measurement data to the processing center and sending back an adjusted glucose measurement result can be cumbersome for each test. In another embodiment, the meter, processing system 26 includes an algorithm for converting raw or preliminary measurement data into an adjusted glucose measurement and, during a given test, generates a result for display without the need for transferring data to the processing center 40. Periodically, say, once a day or once a week, the meter 10 would be connected to the processing center 40 and any new glucose readings would be uploaded to the processing center. Further, any changes in the algorithm(s) used, based on new experience that the processing center 40 has learned since the last update, would be downloaded to the meter. Thus, the glucose meter 10 still benefits from the processing center, but the data would be processed at the meter 10, and thus the user is only occasionally inconvenienced by needing to connect to the processing center 40.

Whether it is raw glucose measurements, preliminary glucose measurements and/or adjusted glucose measurements which are transmitted to the processing center 40, the processing center collects, stores and further processes glucose measurement related data from the meter 10 in order to improve patient care. The result of data processing by the processing center 40 generally falls into the category of (1) a modified method for blood sample processing to provide a highly accurate glucose measurement or (2) patient care instructions. In the case where the processing center provides a modified processing method, the modified method may be performed at the processing center by processor 44 or at the glucose meter by processor 26. As will become apparent from the discussion of FIG. 5 below, the processing performed by the processing center can be automated, partially automated and partially manual, or entirely manual. By manual, it is meant that a trained analyst stationed at the processing center analyzes the data.

As one example of processing performed by the processing center 40 which yields a modification to the blood processing technique, a plurality of glucose measurements from a given patient are monitored over time so that patterns of glucose behavior can be discerned. Glucose readings on personal machines are usually within 20% of the reading that would be obtained from a gold standard sample. However, due to various factors (e.g., too little blood on strip) an occasional reading can be very significantly in error. The processing center 40 analyzes glucose readings for a given person over time and “learns” details about the person's variations over time. After a learning period, algorithms can be implemented to discern which glucose readings are “good” and which are likely artifacts.

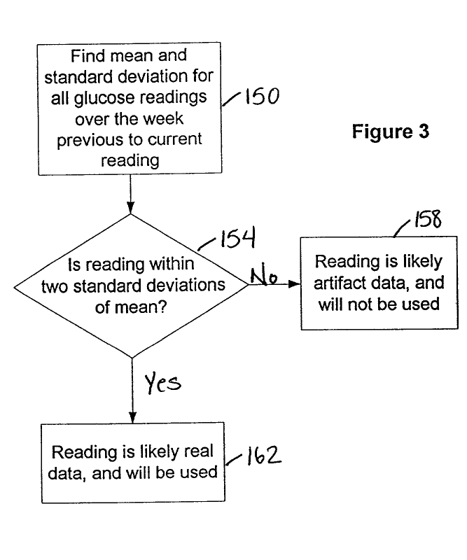

One such process is illustrated in FIG. 3 by which the processing center 40 processes a predetermined number of raw or preliminary glucose measurements of a particular patient in order to derive a likely glucose deviation which yields a likely glucose range for the patient. In step 150, the mean, standard deviation and percentile of glucose readings (generally in the form of raw or preliminary measurements) taken over a predetermined interval, such as the previous week, are computed. In step 154, it is determined whether the current reading is within two standard deviations of the mean value, or within 2.5^{th} and 97.5^{th} percentiles. If the current reading is not within two standard deviations of the mean value, or within 2.5^{th} and 97.5^{th} percentiles, then the reading is considered to be an artifact and the patient is warned about this likely abnormal value as shown in step 158. Alternatively, if the current reading is within two standard deviations of the mean, or within 2.5^{th} and 97.5^{th} percentiles, then the reading is considered accurate in step 162. Thus, in the illustrative example of FIG. 3, the two standard deviations may be characterized as a likely glucose deviation for the patient and, with the mean value, yields a likely glucose range for the patient, with the likely glucose range being between the mean value plus two standard deviations and the mean value minus two standard deviations.

The process of FIG. 3 may be performed each time a new measurement is taken so that the mean and standard deviation values are continuously updated. Alternatively, the process may be performed less frequently and the computed mean and standard deviation values may be used to process some number of future blood samples of the patient by serving as a criteria of acceptability of the future measurements. That is, future glucose measurements of the patient are evaluated to determine if they are within the likely glucose range. If a measurement is within the likely glucose range, then it is accepted as an accurate reading; whereas, if a measurement is not within the likely glucose range, it is considered an artifact and the patient is warned about this likely abnormal value.

It will be appreciated by those of ordinary skill in the art that the process steps of FIG. 3 are illustrative only and can be varied in terms of their order and the manner of implementation. Also, the process might be predetermined and implemented in an automated fashion by software and/or hardware elements at the processing center or physician, analyst, or other input and intervention may be permitted in the process. Also, resulting modified blood sample processing method by which the likely glucose range is used as an acceptance criteria for future glucose measurements may be performed at the glucose meter or at the processing center.

Another type of processing performed by the processing center 40 is related to the characteristics of each lot of test strips 14 (FIG. 1). Each lot of strips 14 has slightly different characteristics, due to manufacturing variations and tolerances. This variation in test strip characteristics is a source of inaccuracy in glucose testing. Typically, manufacturers of glucose meters and test strips compensate for this change between lots of strips by grading each lot of strips based on the lot's properties as measured after manufacture. Conventionally, the grade is entered into the glucose meter in which the strip is used, and the grade is used by the processor 26 in converting the voltage or current signal 18 into a preliminary glucose measurement.

There are several disadvantages to this technique. Strips are graded at the time of, or shortly after manufacture. This grade may provide a somewhat accurate profile of a lot's characteristics at that moment in time, however, the characteristics of a particular lot may well change over time, and that change cannot be communicated to the glucose meter to optimally modify the algorithm at the time the strip is actually used. This change in characteristics between manufacture and use leads to increased inaccuracies in measurement.

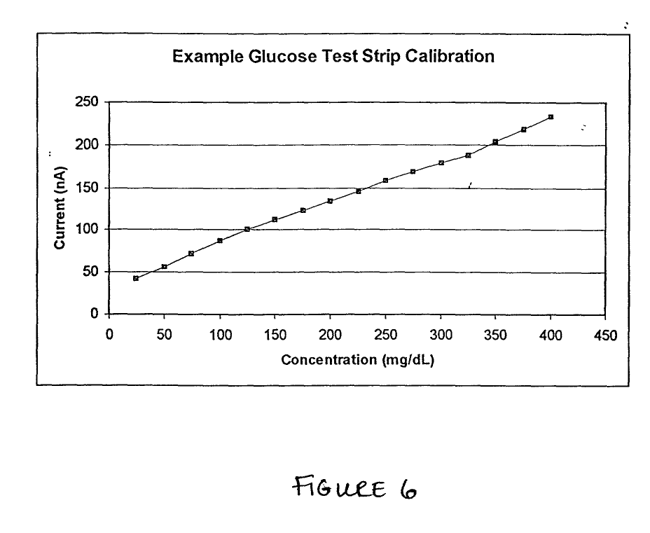

Further, in order to simplify the entry of the lot grade information into the glucose meter, the lots are graded as a single number (e.g. 1-12) or a single letter (e.g. a-o), sometimes referred to as a calibration code. This cannot optimally characterize the characteristics of the lot. According to one aspect of the invention, a polynomial equation including multiple numbers (or coefficients) is used to describe the non-linear nature of the strip's response, as described below in conjunction with FIG. 6.

The processing center 40 monitors how characteristics of a particular lot of test strips are changing over time and uses this information to modify the manner in which further blood samples are processed in order to provide an adjusted glucose measurement. In one embodiment, both the raw glucose signal 18 (or preliminary glucose measurement) and the test strip lot code, either entered by the meter user or read automatically off of the strip, are transmitted to the processing center. The processor 44 accumulates and evaluates the readings from a particular lot of strips over time in order to discern trends. For example, if one lot of strips is tending to read higher over time, this can be discerned by the average values of the many tests read over time. This effort can be enhanced by relating specific patients to specific tests, (say, by including the patient's ID number with the test information) so that a bias is not introduced by a biased patient population that happens to receive a certain lot of strips. Analysis of variance (ANOVA) can be use to demonstrate if the glucose readings are different for each patient for a given lot of strips. In an example using the ANOVA technique where the groups are the patients, for each group will be calculated a mean and a standard deviation, and then the ANOVA will provide a probability (p) value. If this p value is below 0.05, then it means the groups (patients) have different glucose readings, so the differences are due to the patients, but if the p value is high, then it means the groups are equivalent and this lot of strips may tend to have higher or lower values over time. As an alternative way to learn about the aging tendencies of a lot of strips, several strips of known ages can be lab tested with samples having known glucose levels.

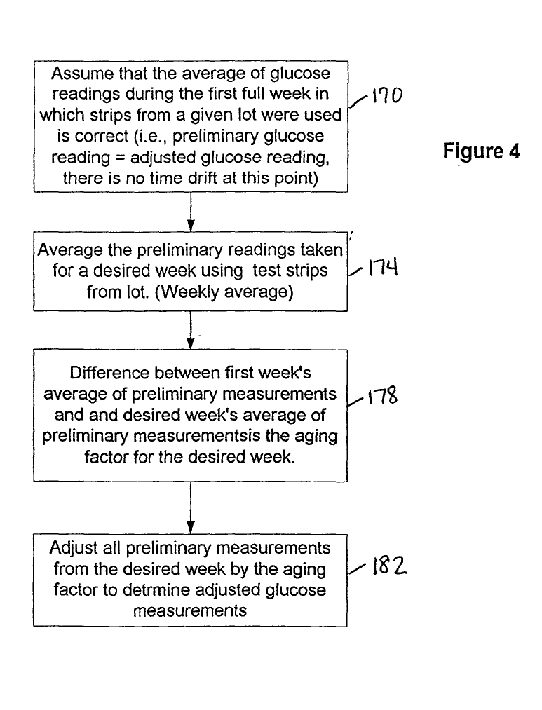

The result of this analysis of actual glucose measurements or laboratory measurements can be described as an aging factor which is a characterization of the lot at the present time, rather than at the time of manufacture. Referring to FIG. 4, an illustrative process for determining the aging factor is shown. In step 170, an assumption is made that the average value of the glucose readings during the first week in which strips from a given lot were used is correct. Stated differently, the average preliminary glucose measurement for the week is set equal to the average adjusted glucose reading since there is assumed to be no drift in readings due to strip aging during the first week of use.

In step 174, the average value of preliminary glucose measurements taken during a predetermined week using strips from the given lot is computed. In step 178, the difference between the first week's average preliminary measurement value and the predetermined week's average preliminary measurement value is computed. This difference value provides the aging factor for the predetermined week.

In step 182, processing of glucose measurements is modified according to the aging factor in order to provide adjusted glucose measurements. Specifically, preliminary glucose measurements taken during the predetermined week are adjusted by adding the aging factor to each preliminary glucose measurement in order to provide corresponding adjusted glucose measurements.

While step 182 of FIG. 4 illustrates that preliminary glucose measurements are processed according to the aging factor, it will be appreciated that raw glucose measurements may alternatively be processed by the aging factor. In this case, raw glucose measurements are used in the assumption of step 170, the averaging of step 174, and the differencing of step 178 as well. It will further be appreciated that an already adjusted glucose measurement may be used in the process steps of FIG. 4. For example, a set of glucose measurements adjusted according to FIG. 3 in order to discard artifact readings may be used to generate the aging factor in step 178 and may be adjusted in step 182 accordingly. Likewise, it will be appreciated that the processing of FIG. 4 may precede the processing of FIG. 3 in order to first adjust glucose measurements based on strip aging tendencies and then adjust the measurements based on the acceptance criteria of the likely glucose range. Although possible, it is not necessary that the both the process of FIG. 3 and the process of FIG. 4 both be performed on a set of glucose measurements.

Glucose readings are just one piece of information that is needed to properly manage the care of a person with diabetes. For example, it is also important to know when a reading was taken with relationship to meals eaten by the person being tested. According to another aspect of the invention, the user interface 24 permits the user to enter data regarding when a meal was eaten. As one example, a button that is pressed whenever a meal is eaten records the time and this information is sent to the processing center 40. The user interface 24 may further permit the patient to enter information regarding the content of the meal and other factors affecting blood sugar levels, such as exercise or activity levels. For example, the relative size of the meal (e.g., light, standard, or large) or the composition of a meal (units of carbohydrates, proteins, and fats) might be entered.

With this information, the processing center 40 can, over time obtain a profile of how the user's blood sugar responds to meals and/or activity levels, and modify the algorithm by which future blood samples are processed at the meter 10 and/or at the processing center 40 in order to compensate for when readings are taken. Glucose levels rise in response to eating, peak, and then decline. It is desirable to control a diabetic patient's medication dosage and timing so as to prevent the peak glucose level from rising too high (a hyperglycemic state). By developing a profile of glucose concentration vs. interval after eating, a profile for a given patient's blood glucose level response over time to a meal can be ascertained. Once this is ascertained, the peak glucose concentration after any meal might be estimated from a reading that is not performed at the time when the peak concentration is expected by using standard estimation methods. The estimated peak value may be displayed to the patient, allowing optimum modification of medications in order to prevent undesirably high peak glucose levels from occurring.

In the category of providing patient instructions, based on the profile by the processing center of how the user's blood sugar responds to meals and activity levels, the meter 10 can be programmed based on data from the processing center to notify the patient of the optimum time to take a reading or the patient can be notified directly by the processing center 40. This can be done by beeping the patient or notifying the patient in some other manner, i.e. a telephone call, e-mail message, etc.

Referring also to FIG. 5, the glucose metering system of FIG. 2 is shown to include a plurality of glucose meters 10a-10n of the type shown in FIG. 1, each capable of communication with the processing center 40 for transmission and receipt of glucose related data, as described above. Also shown is a plurality of physician's computers 70a-70n, each capable of communication with the processing center 40 via the telemetry link 34. With this arrangement, a physician is able to remotely monitor a patient's glucose readings over time and is able to input desired parameters related to the patient's care.

More particularly, a physician may input via a computer 70a-70n criteria upon which it is desired to notify the physician and/or patient. A physician may input a rule that any time a particular patient's blood glucose level is greater than a predetermined level, the physician is to be notified. For example, a physician can register a set of conditions, e.g., a glucose reading higher than 240, that would require action to be taken, e.g., send a message to the physician's pager with the name of the patient, telephone number, and the patient's last five glucose readings. Once this page is received, the physician can discuss the situation with the patient, and appropriate measures can be taken.

Also, a physician may communicate with patients via the processing center 40 by transmitting messages to the processing center for further transmission to the patient via the patient's glucose meter. As examples, this communication mechanism may be used to instruct the patient to modify the patient's treatment regimen (e.g., to instruct the patient to change the glucose dosage) or to modify the glucose monitoring regimen (e.g., to instruct the patient to change the testing frequency and/or times).

The purpose of collecting glucose data and processing the data at the processing center is to understand how a patient's treatment regimen is succeeding or failing, based on the levels of glucose over time. For example, analysis of a patient's glucose readings over time might demonstrate that the patient tends towards hyperglycemia at a certain time of day. This information will permit the patient and physician to alter therapy so as to eliminate the hyperglycemia. Another purpose of collecting glucose data is to flag the occurrence of adverse events, specifically acute hyperglycemic or hypoglycemic events. The latter may require prompt action. For example, high glucose readings may indicate ketoacidosis, which can then be tested for and treated.

In addition to the above-described glucose related data, several pieces of other data can be collected and downloaded to the processing center 40 to improve measurements. For example, meter temperature and other factors that affect the readings can be downloaded as well. Sophisticated algorithms to provide better results can use to this information. Also, these factors can be tracked over time and analyzed to see how various factors are interacting to produce inaccuracies, and thus, better algorithms could be developed and regularly updated. For example, it may be found that the calibration curve in FIG. 6 may only be accurate at a single temperature. Other calibration curves could then be developed to describe a strip lot's performance at different temperatures, and the appropriate curve used based on the meter's current temperature.

Since it is contemplated that the processing center 40 will support many glucose meters and/or physicians through computers 70a-70n, security measures must be implemented by which only authorized access to the processing center is permitted. For example, security features, such as encryption and/or use of a patient ID and password combination, may be used to preserve patient confidentiality.

Data from a plurality of glucose meters 10a-10n collected by the processing center 40 may be combined for use in providing the test strip aging factor described above in conjunction with FIG. 4. That is, glucose tests performed by different patients on different meters, but using test strips from the same manufacturing lot, may be processed to generate the aging factor indicative of how the strips age over time.

As noted above, the telemetry link 34 may be implemented with various hard-wire or wireless transmission media and may include one or more public or private networks. In the illustrative embodiment, the telemetry link 34 is a public telephone system including voice T1 lines and Plain Old Telephone service (POTs) lines. It will be appreciated by those of ordinary skill in the art that the telemetry link may additionally or alternatively include other public or private networks, such as the Internet.

Further in the illustrative embodiment, two types of connections between the telemetry link 34 and the processing center 40 are shown. A first connection 38a is provided by Plain Old Telephone service (POTs) lines coupled to a communication interface in the form of a modem bank 104 and a second connection 38b is provided by a voice T1 line coupled to a communication interface in the form of a Portmaster 90 of the type available from Lucent Technologies which contains modems used to demultiplex the 24 multiplexed voice lines comprising the T1 line. Use of either or both types of connections 38a, 38b is suitable for coupling the processing center to a plurality of glucose meters 10a-10b and physician's computers 70a-70n.

The Portmaster 90 is coupled to a File Transfer Protocol (FTP) server 94 through a non-routable hub 92 (i.e., a hub that has no further connections beyond those shown). A remote access server (RAS) 106 is coupled to the FTP server 94 through a non-routable network interface card (NIC) 108 (i.e., a network card which sees no further network connections beyond those shown). A compressed data file containing glucose related data is transmitted from a glucose meter 10a-10n to the FTP server 94 for temporary storage prior to analysis. Also, in the case where adjusted glucose measurements and/or patient instructions are transmitted from the processing center to the glucose meter, a results file is placed on the FTP server 94 for downloading to the appropriate glucose meter. The illustrative processing center 40 implements the TCP/IP protocol on an Ethernet network.

A traffic cop server 96 implements a database management routine by which the FTP server 94 is monitored for unanalyzed data files and the data files on the FTP server are decompressed and placed on a database server 112 to await analysis. The traffic cop application also monitors the database server 112 for analyzed data so that it can generate results files, compress the results files and place them on the FTP server 94 for downloading by the respective glucose meter. In the illustrative embodiment, the traffic cop server 96 is implemented on a standard Intel x86 compatible server running Windows NT.

The database server 112 contains the glucose related data from the glucose meters 10a-10n and, after analysis, the results generated in response to processing of the glucose related data. The results may take the form of an aging factor indicative of test strip variations over time to be used by the meter processor 26 in converting a raw glucose measurement 18 into an adjusted measurement. The results may be a permissible glucose deviation for a patient to be used as an acceptance criteria applied to a glucose measurement. Additional processing results include patient instructions or notifications from a physician or from the processing center. The results of processing by an analyst workstation may also be an adjusted glucose measurement as may be provided by processing an initial measurement from the glucose meter in response to an aging factor and/or permissible patient deviation for example. In the illustrative embodiment, the database server 112 is implemented with Oracle relational database management system (RDBMS) on an Intel Pentium III-based server running the Windows 2000 operating system.

A plurality of analyst workstations 100a, 100b, . . . 100n are coupled to the database server 112 and may be used by trained analysts to process unanalyzed data from the database server 112 and provide results. Alternatively, or additionally, data analysis may be performed on the analyst workstations by automated or partially automated processes as may be implemented by software routines for example. The results provided by the analysis are packaged by the traffic cop server 96 and placed on the FTP server 94 for downloading by the respective glucose meter.

In the illustrative embodiment, the RAS server 106 implements additional functions including that of a file server, archive server, and backup server. According to its file server functionality, the server 106 contains the analysis software executed on the analyst workstations 100a-100n, as well as other files shared by processing center components. As a backup server, the server 106 periodically creates a snapshot of the data in the database server 112 and the software files in the file server portion of the server 106 in order to permit restoration of data if software files are lost. Data which is not necessary for long-term use (e.g., raw glucose measurements) are periodically moved to the archive portion of the server 106.

A time server 186 is provided for maintaining a master clock with which the time clock maintained in the glucose meters 10a-10n dialing into the processing center 40 can be synchronized. Use of the time server facilitates process time data collection. For example, the time that it takes the processing center to process glucose data and the glucose meter download the results can be monitored.

According to a further aspect of the invention, a more sophisticated expression can be used to characterize the response curve of a test strip as compared to a simple calibration code. For example, a series of coefficients of a polynomial equation that best fits the strip's characteristics can be used. In particular, the precise relationship between current to glucose concentration can be closely described by a polynomial equation such as

C_{G}=a₀+a₁i+a₂i²+a₃i³,

where C_{G} is glucose concentration and i is current.

Referring to FIG. 6, an illustrative response curve for a given a lot of conventional glucose test strip is shown. This particular curve can be well characterized by the following equation:

C_{G}=4E−06i³−0.0029i²+1.0048i+18.753

The coefficients a₀, a₁, a₂, and a₃ may be provided by the strip manufacturer instead of the conventional single number lot grade. The user inputs the coefficients into the glucose meter using for example, a keypad, or the coefficients may be encoded on the test strips when they have been characterized after manufacture and read automatically off the test strip for use in processing raw glucose measurements to provide preliminary glucose measurements. For example, the raw glucose measurement in the form of a current signal is processed according to the above equation to provide an adjusted glucose measurement.

According to a further aspect of the invention, the processing center 40 may be used to prevent manufacturers other than the manufacturer of the glucose meter from making test strips compatible with the glucose meter. Conventionally, in addition to a calibration code, manufacturers put a lot number on packages of test strips in order to identify the manufacturing lot.

In addition to, or instead of the calibration code, the meter user inputs the lot number from the package. In the case where only the lot number is input, the processing center maintains a table relating each lot number of the manufacturer to that lot's updated calibration information. The database of coefficients corresponding to each strip lot may be updated regularly to adjust for changes in strip lots over time, as may be done for example by periodically testing a test strip from the lot to determine the coefficients or by determining an aging factor based on measurement data according to FIG. 4. Once the strip lot number is transmitted to the processing center, the appropriate coefficients may be used to produce the adjusted glucose reading. Alternatively, the appropriate coefficients could be transmitted to the meter and the adjusted glucose reading calculation performed by the meter.

Data can be provided to the processing center 40 regarding how many test strips a particular lot contains. Such data may be input at the processing center itself (e.g., through one of the analyst workstations 100a-100n) or through a manufacturer's computer in communication with the processing center (e.g., like physician computers 70a-70n).

In operation, as the processing center 40 receives glucose measurements, a count is maintained of the number of test strips processed from each lot. Once the total number of test strips of a given lot has been processed, the processing center can expire that lot number, preventing further use or processing of test strips having the same lot number, which may be from a manufacturer of generic test strips who has copied the lot number in order to utilize the processing center. For example, in the case where raw glucose measurements are processed at the processing center to provide an adjusted glucose measurement to the glucose meter, an error message rather than the adjusted glucose measurement can be sent to the glucose meter, thereby preventing use of the test strip. Further use of strips with the expired lot number could be prevented by sending a lockout code to the meter, instructing the meter to not process any more strips with the expired lot number.

According to a further aspect of the invention, each box of test strips can be given a unique box number. A database of the correspondence between box numbers, lot numbers, and coefficients is maintained at the processing center, each lot number corresponding to a plurality of box numbers. The box number for each test strip would be transmitted from meter to processing center, which would maintain a count of the number of strips used from each box. Once the count of strips used from a box reaches the number of strips in the box, the box number is obsoleted and no further strips claiming that box number are processed. To ensure further protection from makers of generic test strips, box numbers may be assigned using an algorithm which ensures that only certain numbers are valid, and thus randomly-assigned or randomly-transmitted box numbers would likely not be accepted by the meter and/or processing center.

It will be appreciated by those of ordinary skill in the art that many modifications can be made to the hardware and software of the processing center 40 without departing from the spirit and scope of the claims. As one example, the processing center may include a pair of redundant processing centers interconnected and operable as described in a pending U.S. patent application No. 09/570,683 entitled MEDICAL TESTING TELEMETRY SYSTEM and assigned to the assignee of the subject invention.

Having described the preferred embodiments of the invention, it will now become apparent to one of ordinary skill in the art that other embodiments incorporating their concepts may be used.

It will be appreciated by those of ordinary skill in the art that the glucose metering system of the invention may include additional collection devices for collecting other physiological data of the patient in order to further enhance the patient's care. For example, the system may include heart rate variability monitors of the type described in U. S. Pat. No. 5,984,954 entitled METHODS AND APPARATUS FOR R-WAVE DETECTION and assigned to the assignee of the present invention. The processing center 40 collects the additional data, such as from the heart rate variability monitor, for providing the physician with a thorough understanding of the patient's condition.

It is felt therefore that these embodiments should not be limited to disclosed embodiments but rather should be limited only by the spirit and scope of the appended claims. All references cited herein are hereby incorporated herein in their entirety.

What is claimed is:

1. A glucose meter comprising:
(a) means for processing a blood sample of a patient to provide a glucose measurement; and
(b) a communication interface to send data including said glucose measurement to a processing center and to receive data from said processing center, wherein said processing means is responsive to said data received from said processing center for modifying the processing of the blood sample to provide an adjusted glucose measurement.

2. The glucose meter of claim 1 wherein said processing means comprises a circuit for analyzing a test strip containing said blood sample to provide said glucose measurement.

3. The glucose meter of claim 2 wherein said data sent to said processing center through said communication interface further includes a calibration code associated with said test strip.

4. The glucose meter of claim 3 wherein said data received from said processing center includes an aging factor indicative of variation in said test strip over time.

5. The glucose meter of claim 1 wherein said data sent to said processing center through said communication interface further includes an identifier of said patient.

6. The glucose meter of claim 5 wherein said data received from said processing center includes a permissible glucose deviation for said patient.

7. A glucose metering system comprising:
(a) a glucose meter for processing a patient's blood sample on a test strip to provide a glucose measurement; and
(b) a processing system coupled to said glucose meter and comprising means for collecting a plurality of said glucose measurements and for processing said plurality of glucose measurements to provide a modified method for processing said blood sample to provide an adjusted glucose measurement.

8. The glucose metering system of claim 7 wherein said modified method for processing said blood sample is performed by said processing system and said adjusted glucose measurement is sent by said processing system to said glucose meter.

9. The glucose metering system of claim 7 wherein said modified method for processing said blood sample is performed by said glucose meter.

10. The glucose metering system of claim 7 wherein said modified method for processing said blood sample is a function of a permissible glucose deviation for said patient.

11. The glucose metering system of claim 8 wherein said modified method for processing said blood sample is a function of an aging factor indicative of variations in said test strip over time.

12. A glucose metering system comprising:
(a) a glucose meter for processing a blood sample on a test strip having a response curve to provide a glucose measurement; and
(b) a processing system coupled to and located remotely from said glucose meter, said processing system comprising means for collecting data including a plurality of said glucose measurements from said glucose meter and a description of said response curve and for processing said data to provide an aging factor indicative of variations in said test strip over time, wherein said aging factor is used to provide said adjusted glucose measurement.

13. The glucose metering system of claim 12 wherein said adjusted glucose measurement is provided by said glucose meter.

14. The glucose metering system of claim 12 wherein said adjusted glucose measurement is provided by said processing system.

15. The glucose metering system of claim 12 wherein said description of said response curve comprises a single number code.

16. The glucose metering system of claim 12 wherein said description of said response curve comprises a polynomial equation.

17. A method for providing a glucose measurement comprising the steps of:
sending a plurality of glucose measurements of a patient from a glucose meter to a remotely located processing center,
evaluating said plurality of glucose measurements to derive a permissible glucose deviation for said patient; and
processing a glucose measurement of said patient in response to said permissible glucose deviation in order to determine whether said measurement is an accurate measurement or an artifact.

18. A method for enhancing the care of a patient using a glucose meter comprising the steps of:
sending data including a glucose measurement and meal information from a glucose meter to a remotely located processing center;
processing said data by said processing center to provide an instruction for the patient; and
sending said instruction to said glucose meter for display to the patient.

19. The method of claim 18 wherein said instruction indicates a time when a glucose measurement should be performed.

20. The method of claim 18 wherein said instruction indicates a medication dosage.
